(19) United States
(12) Patent Application Publication
(10) Pub. No.: US 2009/0269419 A1
(43) Pub. Date: Oct. 29, 2009

(54) COSMETIC COMPOSITION BASED ON POLYUNSATURATED FATTY ACIDS AND ITS USES

(76) Inventors: Jean-Louis Pierrisnard, Oraison (FR); Yves Millou, Valensole (FR); Katia Fontes, Meyrargues (FR); Cecile Tourel, Aix-En-Provence (FR)

Correspondence Address: ARENT FOX LLP, 1050 CONNECTICUT AVENUE, N.W., SUITE 400, WASHINGTON, DC, 20036, US

(21) Appl. No.: 12/307,090

(22) PCT Filed: Jul. 2, 2007
(86) PCT No.: PCT/FR07/01115

§ 371 Date: Apr. 15, 2009

(30) Foreign Application Priority Data
Jun. 30, 2006 (FR) 06/05953

(51) Int. Cl. A61K 33/34; A61K 36/00

(52) U.S. Cl. 424/638; 424/725

(57) ABSTRACT

The present invention relates to a cosmetic or dermatological composition comprising (i) at least vegetables oils which are rich in omega-3 and/or in omega-6, selected from the group consisting of echium oil, cameline oil, evening primrose oil and borage oil, the vegetable oils being present in a proportion such as to provide an omega-3 to omega-6 ratio within said composition of between 1 and 5, (ii) at least one mineral, preferably in salt form, selected from the group consisting of sodium, calcium and magnesium, preferably magnesium, (iii) at least one trace element selected from the group consisting of zinc, copper and iron, preferably from the group consisting of zinc and copper, (iv) at least vitamin C or derivative thereof, and (v) at least one essential oil, which has anti-free-radical properties, for performing a deep-down treatment which stimulates the microcirculation and the production of energy for the purpose of imparting dynamism to the cells and accelerating their renewal, protecting the collagen, and combating pigmentation spots.

The present invention relates to a cosmetic or dermatological composition which neutralises free radicals and stimulates the microcirculation and the production of energy in order to revitalise the cells and to accelerate their renewal, to protect the collagen and to combat pigmentation marks. More particularly, the invention relates to a cosmetic or dermatological composition comprising at least oils which are rich in omega-3/omega-6, such as echium, camelina, evening primrose and borage, trace elements such as zinc, copper and magnesium, a vitamin C derivative and at least one essential oil, preferably a Helichrysum essential oil.

The conditions of modern life subject the organism to various constant attacks (pollution, heavy metals, extreme temperatures, UV, etc.).

The skin, which constitutes a physical, thermal but also immunological barrier between the body and the external elements, therefore has to face these various attacks. Among the latter, one major attack results from the action of free radicals, the chemical reactivity of which causes a change in the various cell components of the skin.

However, the ageing of the skin impairs its efficacy. This is because said ageing slows down the cell renewal of the skin and of its extracellular matrix, in particular the renewal of type I collagen. This reduced rate of renewal increases the sensitivity of the skin to external attacks and its deterioration over time.

Although various compositions have been developed in the prior art, none provides a complete answer for combating all the signs of ageing and simultaneously exhibiting an action of:
stimulating the proliferation of keratinocytes in order to repair the tissue, which proliferation is accelerated by stimulating the production of energy and the microcirculation of the skin;
stimulating the production of collagen and exhibiting an anti-collagenase action for a firmer skin; and
inhibiting the formation of pigmentation marks.

Still today, therefore, there is a considerable need to identify new compositions having an overall action on the skin.

Fats are highly calorific and sometimes dangerous, but some of them are entirely beneficial to health and today we know that they are substances which are essential for the correct functioning of the organism. Fats thus serve as an energy store, they participate in the architecture of living structures, they form part of chemical messengers: life is impossible without them. Furthermore, they play a major role at skin level since, when the fatty acids of the cells are damaged by free radicals, the membranes wither and display as the first clinical symptom a dry and dull skin.

The energy role of fats is significant since, for an equal weight, they provide more calories than sugars or proteins. They are therefore a basic material which makes it possible to store energy in a minimum amount of space.

Fats also play a major role in maintaining the integrity of the body: 15% of the weight of the body consists of fats which have the function of securing the organs and keeping them in place.

They are therefore indispensable for the development of the brain and the maturing thereof. They make it possible to store body heat and to avoid sudden fluctuations in temperature. They supply a large part of the fatty acids and fat-soluble vitamins which the body necessarily requires in order to build itself up and remain in good health.

Fats ensure the absorption of vitamins A, E, D, K, said vitamins being referred to as fat-soluble. They are the main source of essential fatty acids (EFAs) which the organism does not know how to synthesise.

In the 1930s, the relationship was first discovered between skin problems such as certain eczemas and a deficiency of so-called essential fatty acids, which were initially referred to as vitamin F. This name, which was abandoned in some countries, had the merit of underlining the importance of this fatty element within the skin.

Essential Fatty Acids are also involved in the balancing of the nervous system, hormonal and metabolic regulation, blood flow, sexuality, and the quality of the skin and integuments (hair and nails).

In principle, as is the case for sugars and cholesterol, there are “bad” fatty acids known as “saturated” fatty acids, which are responsible for many health problems, and “good” fatty acids known as “unsaturated” fatty acids, which on the contrary have a protective action.

More specifically, the fats are split into four groups:
saturated fatty acids;
monounsaturated acids (still called omega-9 since their only double bond is located on the 9th carbon of their molecule);
polyunsaturated acids omega-6 (indicating the site of a chemical double bond on the 6th carbon of the fatty acid, which contains between 4 and 26 carbon atoms) and
polyunsaturated acids omega-3 (indicating the site of a chemical double bond on the 3rd carbon of the fatty acid).

Among these various fatty acids, two are referred to as “essential”:
linoleic acid (omega-6); and
alpha-linolenic acid (omega-3).

This is because the other fatty acids derive from these two essential fatty acids, which must be present in sufficient quantity and have a balanced ratio. Thus, if one of these two fatty acids is present in too large a quantity, its transformation will be to the detriment of the other. By way of example, an excessive consumption of sunflower oil promotes the omega-6 pathway to the detriment of the omega-3 pathway, which gives rise to an overproduction of pro-aggregating substances and increases the risk of vascular thrombosis. A balanced supply of omega-3 and omega-6 is therefore essential in order to maintain a body balance. According to the Conseil Supérieur de l'Hygiène, this omega-3/omega-6 ratio is from 1 to 4-6, according to the WHO (World Health Organisation) it is from 1 to 5 and more, and according to the NIH (National Institute of Health) it is from 1 to 3 and more. By way of example, breast milk has a ratio of 1 to 5.

It is admitted by the scientific community that essential fatty acids and more particularly polyunsaturated fatty acids (PUFAs) play a fundamental role in the construction and functioning of cells. They constitute a part of the membrane and ensure the sealing of the latter and the exchanges with neighbouring cells. They stimulate the defences of the organism; they improve the learning ability and memory and are involved in the quality of vision; they nourish the skin, giving it a better appearance.

Plant oils which are rich in polyunsaturated fatty acids are in perfect affinity with the skin, promoting the vital processes thereof: they stimulate in particular the formation of the dermo-protective film which makes the skin smooth and supple.

A plant which has for a long time been forgotten, echium is returning to the forefront of medicinal plants. Its high content of Essential Fatty Acids (EFAs), particularly of a-linolenic acid, justifies its medicinal and cosmetic properties. From the Boraginaceae family, echium is a plant with an erect stem, highly floriferous, having the particular feature of being entirely covered with stiff and prickly hairs, the flowers are arranged in clusters and have a corolla of 1.5 to 2 cm, flowering taking place from May to July. This plant, which grows wild in arid locations, can measure 1 to 3 metres in height. One particular feature of echium is the change in colour of its flowers depending on the level of acidity of the cellular sap. The young flower has an acidic red sap, while the ageing flower has an alkaline blue sap. Echium oil is distinguished from other plant oils by its high percentage of stearidonic acid which is found mainly in the oils of coldwater fish.

Camelina (Camelina sativa or German sesame) is a herb originating from the steppes of Asia. Cultivated in Europe since pre-history, the culturing thereof has become more rare. An annual plant from the Cruciferae family, of rapid growth, it grows at an altitude of up to 1200 m. Its rigid, straight stem reaches a height of 1 m. Its more or less hairy leaves end in two pointed ears. Small yellow flowers with 4 petals in the shape of a cross blossom from May to July. At the end of summer, the clusters of flowers bear fruits in the form of small pears, filled with minuscule reddish seeds.

Another plant which is rich in EFAs, evening primrose, of the Onagraceae family, is a plant with an erect, angled stem which bears large yellow flowers. The stem carries many fruits. Biannual evening primrose generally grows in sunny or partially shaded conditions in silicious soil. Its leaves are single, long and downy, with veins. The flower contains 8 stamens, 4 petals of 4 to 5 cm which are shorter than the 4 sepals. This flower opens at the end of the day, hence its common name of evening primrose. Flowering takes place from June to September, after which the fruit appears, which measures from 2 to 3 cm.

Another plant which is rich in EFAs is borage (Boraginaceae family) which is an annual plant which can reach 60 cm and which is covered with stiff and prickly off-white hairs. Its alternate leaves are matt green, fleshy and wavy with sessile upper leaves hugging the stem by their base. The flowers are borne by a long hairy stalk in a star shape with five pointed petals of deep blue when mature and pink when very young. The fruits consist of four brown or blackish achenes with grooves.

Organic or mineral elements (generally in the form of salts), mainly calcium, sodium and magnesium, and trace elements (iron, zinc, copper, etc.) also play an important physiological role, mainly acting on the cellular enzymatic activity where they participate as a co-factor in prosthetic groups of enzymes.

At the skin surface, mineral salts are found in the keratinocytes, cells of the epidermis, but also in the fibroblasts which belong to the cells of the dermis. Mineral salts form part of the composition of NMF (Natural Moisturising Factor).

More specifically, magnesium is an important intracellular cation and an activator of many enzymatic systems. It is a mineral which is essential for maintaining the good health of the organism. Magnesium is involved for instance in the phosphate transfer system and in the energy production system (synthesis of ATP). Age brings with it a deficiency of magnesium in the skin. Since magnesium participates in many metabolic reactions, magnesium supplementation is necessary in order to keep the skin in good physiological condition.

The trace elements are represented at the hydro-lipid-protein film, hence their role in maintaining skin hydration.

Copper is an important trace element since it is involved as a co-factor in many oxidation reactions. For instance, copper is involved in oxidative metabolism and in cellular respiration, where it allows the synthesis of ATP due to its role as co-factor in various enzymatic reactions. Copper also has an effect on cell vitality, which is linked to its functions in protein synthesis. At the skin level, copper is involved in the synthesis of keratin, a major protein for supporting the epidermis, hair and nails, where it acts as a catalyst for the formation of disulphide bridges. Copper also stimulates the production of collagen, a structural protein of the dermis, and a copper deficiency causes a reduction in the formation of intramolecular bonds, leading to a decrease in collagen synthesis. Finally, copper is involved in cell defence mechanisms and more particularly at the level of the anti-free radical enzymes such as superoxide dismutase (SOD).

Zinc is also an important trace element since it is necessary in particular for good cell growth with an action on the endocrine systems but also as a bio-catalyst in enzymatic reactions, particularly at the dermal level. The skin contains a significant proportion of the zinc in the body (approximately 20%) and a zinc deficiency is associated with significant desquamation. A co-factor of zinc-dependent enzymes, zinc stimulates the rebuilding of the collagen matrix and encourages the phenomena of healing and dermal collagen synthesis. Zinc also makes it possible to combat the phenomena of ageing by stabilising proteins and stimulating the expression of genetic material. Zinc is involved for instance in the synthesis of keratin by contributing to the formation of intra-molecular bridges. By promoting the synthesis and stability of this structural protein, zinc maintains the epidermis and the capillary fibre.

Important research carried out by the Applicant has shown that a composition comprising (i) plant oils (echium, camelina, evening primrose and borage), with a ratio of omega-3 to omega-6 of between 1 and 5, (ii) trace elements (magnesium, copper and zinc), (iii) a vitamin C derivative (tocopheryl acetate), and (iv) an essential oil (Helichrysum) has a synergistic and overall effect on the skin.

Consequently, a first subject matter of the invention consists of a cosmetic or dermatological composition, characterised in that it comprises:
(i) at least plant oils which are rich in omega-3 and/or in omega-6, selected from the group comprising echium, camelina, evening primrose and borage oils, which plant oils are present in a proportion which makes it possible to obtain a ratio of omega-3 to omega-6 in said composition of between 1 and 5,
(ii) at least one mineral, preferably in salt form, selected from the group comprising sodium, calcium and magnesium, preferably magnesium,
(iii) at least one trace element selected from the group comprising zinc, copper and iron, preferably from the group comprising zinc and copper,
(iv) at least vitamin C or a derivative thereof, and
(v) at least one essential oil, which has anti-free radical properties, preferably a Helichrysum essential oil.

The composition according to the invention makes it possible to stimulate the production of energy and the proliferation of keratinocytes, to neutralise free radicals, to stimulate the microcirculation and therefore to carry out a deep treatment.

In the context of the present invention, “rich” in omega-3 and/or in omega-6 is understood to mean an oil having an omega-3 and/or omega-6 concentration greater than 10%.

Using only his general knowledge and by means of routine experiments, the person skilled in the art will be able to identify plant oils which are rich in omega-3 and/or in omega-6. Likewise, the person skilled in the art will easily be able to adjust the proportion of each of these oils in order to obtain a ratio of omega-3 to omega-6 in said composition of between 1 and 5. For instance, and by way of example:
a) Echium oil and more specifically echium seed oil has an average concentration of 30% alpha-linolenic acid (omega-3).
b) Camelina oil and more specifically camelina seed oil has an average concentration of 35% alpha-linolenic acid (omega-3).
c) Evening primrose oil and more specifically evening primrose seed oil has an average concentration of 10% gamma-linolenic acid (omega-6) and 70% linoleic acid.
d) Borage oil and more specifically borage seed oil has an average concentration of 19% gamma-linolenic acid (omega-6) and 35% linoleic acid.

According to a first preferred embodiment, the composition according to the invention comprises echium, camelina, evening primrose and borage oils and particularly preferably echium seed, camelina seed, evening primrose seed and borage seed oils.

The plant oils of echium and camelina which are rich in omega-3 and of evening primrose and borage which are rich in omega-6 have been selected on account of their relatively constant content of active principle, which makes it possible to easily adjust the percentages in order to obtain the omega-3/omega-6 ratio of between 1 and 5. Furthermore, the supply of antioxidants and fatty acids provided by borage, evening primrose, echium and camelina oils constitutes an effective measure for recovering or obtaining rapidly a beautiful skin. In the composition according to the invention, echium oil, due to its composition of EFAs, actively stimulates the regeneration of cell membranes and effectively combats drying of the skin.

The person skilled in the art will be able to determine without difficulty the quantities of the various components necessary in order to obtain the desired effect.

Advantageously, the composition according to the invention comprises a content of:
a) echium oil of between 0.1% and 10% of the total weight of the composition, preferably between 0.1 and 5%, and/or
b) camelina oil of between 0.1% and 10% of the total weight of the composition, preferably between 0.1 and 5%, and/or
c) borage oil of between 0.1% and 10% of the total weight of the composition, preferably between 0.1 and 5%, and/or
d) evening primrose oil of between 0.1% and 10% of the total weight of the composition, preferably between 0.1 and 5%.

Essential oils are complex assemblies of odorous products taken from various aromatic plant organs (roots, flowers, fruits, leaves) by distillation or expression, preferably by distillation. Using his general knowledge, the person skilled in the art will be able to identify without difficulty the essential oils which have anti-free radical properties.

According to a second preferred embodiment, the composition according to the invention comprises a Helichrysum essential oil (Helichrysum italicum), preferably a Helichrysum oil obtained by steam distillation from flowering tops.

Advantageously, the composition comprises a content of Helichrysum essential oil of between 0.05% and 5% of the total weight of the composition, preferably between 0.05 and 2.5%.

According to a third preferred embodiment, the composition according to the invention comprises zinc, copper and magnesium.

This is because it appears that these three components stimulate the cells by increasing the energy balance (ATP) and the basal metabolism.

By way of example, zinc, copper and magnesium are added to the composition according to the invention in the form of a cocktail of minerals having an energising action “Sepitonic M3” available from SEPPIC.

Advantageously, the composition according to the invention comprises a content of:
a) magnesium of between 0.001% and 5% of the total weight of the composition, preferably between 0.001 and 1%, and/or
b) copper of between 0.001% and 5% of the total weight of the composition, preferably between 0.001 and 1%, and/or
c) zinc of between 0.001% and 5% of the total weight of the composition, preferably between 0.001 and 1%.

Vitamin C (ascorbic acid) and its derivatives are well known to the person skilled in the art.

Advantageously, the composition according to the invention comprises a content of vitamin C or one of its derivatives of between 0.5% and 10% of the total weight of the composition, preferably between 0.5% and 5%.

According to a fourth preferred embodiment, the composition according to the invention comprises, as the vitamin C derivative, Ascorbic Acid 2-Glucoside (or O-α-D-glucopyranosyl-(1→2)-L-ascorbic acid).

The Applicant has in fact demonstrated that the addition of such a vitamin C derivative made it possible on the one hand to reduce the melanin already produced and on the other hand to inhibit the production of new melanin.

A vitamin C derivative stabilised with glucose combines with an enzyme present in the skin, alpha-glucosidase, to allow a delayed release of the active ingredient. This slow release will reduce age-induced pigmentation marks by inhibiting the production of melanin.

Such a vitamin C derivative is available in particular from HAYASHIBARA.

The compositions according to the invention may be in all the galenic forms conventionally used, and well known to the person skilled in the art, in the cosmetic or dermatological fields for application to the skin, such as for example oil-in-water (O/W) or water-in-oil (W/O) emulsions, lotions, sprays, gels, milks, creams, etc., without any particular galenic restriction other than those relating to the compatibility of the composition with the excipient.

Furthermore, the compositions of the invention may also contain all the constituents conventionally used in these presentations as formulation agents, preserving agents, solubilising agents, gelling agents, surfactants, fatty substances, propylene glycol, sorbitol, glycerol, perfume bases and water.

The composition according to the invention may furthermore comprise at least one other active ingredient selected from the group comprising refining, restructuring, anti-inflammatory, hydrating and antiseptic agents for the skin.

Advantageously, the composition according to the invention then comprises a content of said active ingredient of between 1 and 20% of the total weight of the composition, preferably between 1 and 10%.

A second subject matter of the invention is a cosmetic care method comprising a step of applying to the skin of a subject an effective quantity of a composition as defined above.

The care method according to the invention makes it possible to stimulate the production of energy, cell proliferation, the neutralisation of free radicals and the microcirculation, to stimulate and protect the production of collagen, and to reduce and inhibit the production of melanin.

A third subject matter of the invention corresponds to the cosmetic use of a composition as described above for carrying out a deep treatment which stimulates the production of energy, revitalises and accelerates cell renewal and inhibits the production of melanin.

The composition according to the invention protects the skin against attack from free radicals and slows ageing.

Other features and advantages of the present invention will become apparent on reading the following non-limiting examples.

### EXAMPLE 1

### Complete Cream—Multiaction Face Care

| | |
|---|---|
| Sucrose stearate | 0.5 to 6% |
| Sucrose distearate | 0.5 to 6% |
| Caprylic/capric triglyceride | 3 to 15% |
| Caprylic/capric/succinic triglyceride | 3 to 15% |
| Echium oil | 0.1 to 5% |
| Camelina oil | 0.1 to 5% |
| Borage oil | 0.1 to 5% |
| Evening primrose oil | 0.1 to 5% |
| Magnesium | 0.001 to 1% |
| Copper | 0.001 to 1% |
| Zinc | 0.001 to 1% |
| Helichrysum essential oil | 0.05 to 2.5% |
| Ascorbic Acid 2-Glucoside | 0.5 to 5% |
| Tocopheryl acetate | 0.05 to 1% |
| Urea | 0.5 to 2% |
| Glycerin | 0.5 to 3% |
| Sodium hyaluronate | 0.5 to 3% |
| Potassium sorbate | 0.2 to 0.5% |
| Xanthan gum | 0.1 to 0.5% |
| Perfume | qs |
| Preservatives | qs |
| Water | qs for 100% |

### EXAMPLE 2

### Anti-Free Radical Activity

### 1) Aim of the Study

The hydroxyl radical is a highly reactive molecule, the production of which is activated by the various stresses to which the skin is subjected (pollution, UV, change in temperature, etc.). This radical is responsible for various types of damage in the cell and in particular for the degradation of the membranes due to the phenomenon of lipid peroxidation.

The aim of the present study was to assess the anti-free radical power of Helichrysum essential oil (Helichrysum italicum) by the in vitro conjugated diene method. This method makes it possible to test the protective effect of said essential oil on liposomes (membrane analogues) subjected to attack by hydroxyl radicals (OH—),

### 2) Principle of the Study

The effect of the free radicals on the polyunsaturated fatty acids of the membranes corresponds to a peroxidation leading to the formation of conjugated dienes which absorb at 233 nm.

This peroxidation is therefore characterised by an absorption peak at 233 nm.

The activity of the antioxidants can therefore be assessed by their ability to prevent the peroxidation of polyunsaturated fatty acids contained in liposomes (in vitro model coming closest to the reality in vivo).

### 3) Materials and Methods

### Reagents:

Phosphate buffer 0.05 M pH 6.8 (Na2HPO4: 3.55 g; KH2PO4: 3.4 g; H2O qs for 1000 ml)
Potassium thiocyanate solution 0.03 M (KSCN: 0.3 g; phosphate buffer qs for 100 ml)
Liposome suspension (Natipides: 3 g; Glydant (preservative): 0.3 g; Distilled water qs for 100 ml)
FeCl₂ solution 2.3×10⁻³ M
H₂O₂ solution, 110 volumes diluted to 10^{th}
CaCl₂ solution 0.5 M
Heptane

### Protocol:

The activity of a solution comprising 0.1% Helichrysum essential oil was metered simultaneously with a control solution. After homogenisation, the solutions were left in an incubator at 45° C. overnight. 5 ml of each solution were then taken as a sample. The lipids were precipitated by adding 5 ml of CaCl₂.

This mixture was stirred for 10 minutes using a magnetic stirring bar, then the lipids were extracted by adding 10 ml of heptane and by stirring for 15 minutes.

The UV spectrum (200 to 350 nm) of each solution was then recorded against heptane, in quartz cuvettes. A peak at 233 nm demonstrates the presence of conjugated dienes and makes it possible to quantify the latter compared to the control.

### Results:

At the end of the test, it appears that the Helichrysum essential oil product (Helichrysum italicum) inhibits lipid peroxidation by 82%±2 under experimental conditions.

### EXAMPLE 3

### Stimulation of ATP Synthesis

Cell energy, which is necessary for any activity, exists in the form of ATP. The production of ATP is ensured in part by the degradation of simple carbohydrates, such as glucose, into pyruvate (glycolysis).

### 1) Principle of the Test

After incubation for 6 hours, the intracellular ATP content was measured by bioluminescence using the kit “ATP Monitoring Kit” (LABSYSTEMS) according to the manufacturer's instructions. The results were expressed in mM of ATP and as a percentage of the control group.

Effect of Sepitonic M3 and of cytochrome C on the ATP content of keratinocytes after incubation for 6 hours:

| | |
|---|---|
| Cytochrome C 0.01%: | ES 110% |
| Sepitonic M3 0.001%: | ES 128% |

The pyruvate initially produced is transformed into acetyl-CoA and the latter, via the citric acid cycle established in the mitochondria, produces a large quantity of NADH, H+. During cell respiration, the NADH, H+ serves as an electron donor source. Along the entire electron transport chain (and oxidative phosphorylation chain), the energy released allows the synthesis of ATP.

In conclusion, Sepitonic M3 revitalises the cells by stimulating the cell energy metabolism with an efficacy greater than the reference at a concentration of 10 times less. The production of ATP in the keratinocytes is then increased by 32% compared to the control. This “additional” ATP constitutes a reserve of additional energy for improving the proper functioning of the cells.

### EXAMPLE 4

### Exploration of the Proliferative Activity of an Oily Omega-3 and Omega-6 Complex on Epidermal Structures after Multiple Applications on Explants of Human Skin Kept Alive

### 1) Aim of the Study

The aim of this study is to explore the proliferative activity of an oily complex on explants of human skin kept alive.

The activity was evaluated by histological expertise from the general morphology of the skin after staining with Masson's trichrome and by immunolabelling of the cells in mitosis (KI 67) (mitotic index).

### 2) Operating Method

### Preparation of the Explants

Preparation of 15 explants of human skin and survival in specific medium. The explants were split into 2 batches of six explants and one control batch TO of three explants, as follows:

| | | |
|---|---|---|
| T0 | Control plasty | 3 explants |
| T | Untreated control | 6 explants |
| P1 | Explants treated with the complex to be tested | 6 explants |

### Treatment

Treatment was carried out on D0, 2 hours after the preparation of the explants, then on D1, D2, D4, D6 and D8.

The products were applied to the explants as follows: T the explants did not receive any treatment, P1 each of the explants received 25 ml of the oily complex deposited on a paper disc and applied to each explant.

The treatment was carried out by topical application of the product to be tested. The product was then spread across the entire surface of the explant, using a spatula. Half of the culture medium was renewed every two days, and the explants were kept alive at 37° C. in a humid atmosphere enriched with 5% CO2.

### Sampling for Histology

On D3 and D9, 3 explants from each batch were taken as a sample. The samples were cut into two, one half was fixed in ordinary Bouin's solution and the other half was frozen at −80° C.

### Morphological Study

After 24 hours of fixing in ordinary Bouin's solution, the samples were dehydrated, impregnated with paraffin, placed in blocks, and sections of 5 μm were formed. The morphological study of the epidermal and dermal structure was carried out on the sections stained with Masson's trichrome, Goldner variant.

### KI 67 Immunolabelling

On the frozen samples, 7 mm sections were formed. The immunolabelling of the cells in mitosis was carried out on frozen sections using anti-KI 67 polyclonal antibody (Novo Castra) revealed with DAB (DiAmino Benzedine).

The positive cells were counted along the epidermal length and the averages were taken as the number of labelled cells per centimetre.

### Results

### General Morphology

Control T0: The stratum corneum is thick, compact, quite markedly keratinised at the surface and at its base. The epidermal structure has 4 to 5 cell layers exhibiting a good morphology. The relief of the dermo-epidermal junction is pronounced. The papillary dermis exhibits a collagen which is more or less fine, quite dense and well cellularised.

Control on D3: The stratum corneum is thick, very slightly flaky, moderately keratinised at the surface and slightly at its base. The epidermal structure has 5 to 6 cell layers exhibiting a good morphology. The relief of the dermo-epidermal junction is pronounced. The papillary dermis exhibits a collagen which is more or less fine, more or less dense and well cellularised.

Omega-3/omega-6 fatty acid complex on D3: The stratum corneum is thick, quite compact, moderately keratinised at the surface and slightly at its base. The epidermal structure has 3 to 4 cell layers. The relief of the dermo-epidermal junction is pronounced. The papillary dermis exhibits a collagen which is more or less fine, not very dense and well cellularised.

Control on D9: The stratum corneum is thick, quite compact, slightly keratinised at the surface and more markedly at its base. The epidermal structure has 4 to 5 cell layers exhibiting a good morphology with slight spongiosis. The relief of the dermo-epidermal junction is pronounced. The papillary dermis exhibits a collagen which is more or less fine, quite dense and well cellularised.

Omega-3/omega-6 fatty acid complex on D9: The stratum corneum is thick, quite flaky, markedly keratinised at the surface with moderate parakeratosis. The epidermal structure has 4 to 5 cell layers. The relief of the dermo-epidermal junction is pronounced. The papillary dermis exhibits a collagen which is more or less fine, more or less dense and well cellularised.

### Cells in Mitosis:

The results of the counts of the cells in mitosis are shown in the following table:

| | Control | Complex |
|---|---|---|
| D0 | 180 | 180 |
| D3 | 25 | 80 |
| D9 | 25 | 20 |

### Discussion

### General Morphology:

On D3 and D9, the epidermal structure is normal on the untreated explants.

On those treated with the omega-3/omega-6 fatty acid complex, compared to the untreated explants, the stratum corneum is markedly flaky all over on D9. The epidermal thickness is thinner on D3 and comparable on D9.

### Mitotic Index:

On D3, the mitotic index shows a very marked increase in the number of cells in mitosis on the explants treated with the omega-3/omega-6 fatty acid complex compared to the control explants.

### Conclusion

Under these operating conditions, the omega-3/omega-6 fatty acid complex shows on D3 a marked stimulating activity on the keratinocytes.

The control skin kept alive has a normal decrease in cells in mitosis. The stimulation is measured by the ratio between 80 and 25.

Without the active ingredient, this reduction is 86%. With the oily complex, it is just 56%. It is possible to deduce from this that there is a 30% stimulation of cell division after 3 days.

### EXAMPLE 5

### Effect of Ascorbic Acid 2-Glucoside (AA-2G) on the Reduction of Melanin

### 1) Method

a) Culturing of melanocytes for 48 hours
b) Addition of AA-2G 2 mM for 48 hours
c) Lysis of the cells in order to read the quantity of melanin contained.

### 2) Results:

The results showed a 60%±2 reduction of intra-cellular melanin in the presence of AA-2G.

### EXAMPLE 6

### Inhibition of Melanogenesis by AA-2G

### 1) Method

a) Culturing of melanocytes with AA-2G 2.5 mM+control without AA-2G for 12 hours
b) Stimulation of melanogenesis by theophylline (0.5 mM)
c) Detection of melanin by colorimetry (photos).

### 2) Results:

The photos of the treated melanocytes showed a lesser quantity of developed melanin than on the untreated melanocytes.

### EXAMPLE 7

### Evaluation of the Anti-Collagenase Activity

### 1) Aim of the Study

The aim of this study is to demonstrate the anti-collagenase activity of an essential oil and of a formulation containing an essential oil.

### 2) Operating Method

The anti-collagenase activity test was carried out on ds-es frozen sections. The products to be tested were mixed (volume/volume) with a collagenase solution at 60 U/ml. Incubation took place in a humid chamber for 2 h at 37° C. The remaining collagen was stained with picro-sirius and quantified by image analysis.

### Batch:

1. Control TRIS buffer
2. Collagenase 30 U/ml
3. Reference R: phenanthroline
4. Reference R: phenanthroline+collagenase 60 U/ml
5. Product P2: Immortelle very precious cream ref 05.34.A
6. Product P2: Immortelle very precious cream ref 05.34.A+collagenase 60 U/ml

### 3) Histological Examination

Control TRIS Buffer:

The collagen observed is well structured.

Collagenase:

The collagen observed is highly unstructured.

Phenanthroline 1.25%:

The collagen observed is well structured.

Phenanthroline 1.25%+Collagenase:

The collagen observed is well structured.

Immortelle Very Precious Cream (0.12% Helichrysum):

The collagen observed is well structured.

Immortelle Very Precious Cream+Collagenase:

The collagen observed is well structured.

### 4) Measurements of the Staining by Image Analysis

For each sample, 4 microscopic fields were analysed as a function of the size of the sample. Each microscopic field is digitised and analysed according to the method described below using the LEICA QWIN software.

1. Digitised Image
Staining of the collagen network

2. Detection of the Collagen by Thresholding
Binary mask 0

3. Determination of the Zone of Interest
Exclusion of the epidermis and annexes
Inversion of the mask so as to retain only the zone corresponding to the dermis
Binary mask 2

4. Selection of Collagen in the Zone of Interest
Binary mask 3=2+0

5. Surface measurement Measurement of binary mask 3
Measurement of binary mask 2

6. Results Exporting of results to Excel
Calculation of the percentage of surface area occupied by collagen in the dermis

The results obtained as a percentage of surface area occupied by collagen in the dermis

| | Without collagenase | With collagenase |
|---|---|---|
| | Mean (typical | Mean (typical |
| | variance) | variance) |
| Control (TRIS | 88.3 (2.9)< | 41.6 (2.1) |
| buffer) | | |
| Positive reference | 88.7 (1.5) | 90.4 (3.8) |
| (phenanthroline | | |
| 1.25%) | | |
| P2 Immortelle very | 86.0 (4.4) | 88.0 (2.9) |
| precious cream | | |

### Discussion

Phenanthroline, tested at 1.25% as positive reference, completely inhibits the effect of collagenase, thus validating the study. The results show that Immortelle very precious cream completely inhibits the effect of collagenase in a proportion close to phenanthroline.

### Conclusion

Under the operating conditions described above, the product “Immortelle very precious cream” has a strong anti-collagenase activity.

### EXAMPLE 8

### Study of the Effect of the Composition on the Synthesis of Type 1 Collagen

### 1) Introduction:

The fibres of the dermis: The fibres of the dermis comprise collagen fibres and elastic fibres. In quantitative terms, they represent the most important structural proteins of the dermis, i.e. respectively 75% and 5% of their dry weight. Their relative proportion and their arrangement are different depending on the surface zones or depths of the dermis.
The fibroblasts: The fibroblasts are responsible for the synthesis and maintenance of extracellular material. These are cells of mesenchymatous origin which synthesis collagen, elastin, ground substance and structural glycoproteins.
The collagen fibres: The collagens form a very large family. These are molecules of the extracellular matrix composed of three polypeptide chains carrying the repetition of three amino acids: -Gly-X-Y, where X and Y are often prolines or hydroxyprolines. The collagen fibres of the dermis consist respectively of collagen I and collagen III, around an axis composed of collagen V. These collagens belong to the group of fibrillar collagens. In adults, collagen I is on average six times more abundant than collagen III.
Collagens and ageing: The collagen I/collagen III ratio decreases in the course of ageing. There is an exponential increase in chemical bridging between the collagen fibres due to Maillard's non-enzymatic glycation reaction. This chemical bridging of the collagen results in a rigidification of the fibres. The degradation and renewal thereof are thus slowed.
Modification of the fibroblasts: The fibroblast is a key cell in the connective tissue which is involved in the formation and stabilisation of elastic fibres, but also in the dystrophy and lysis thereof. During ageing, the fibroblast decreases its activity and this cell at rest is called a fibrocyte. It becomes globular with a reduction in its cytoplasm and a rarefaction of its endoplasmic reticulum, the vesicles of which are very dispersed. This cell is no longer in contact with the collagen.

### 2) Principle of the Study

The modification which promotes the loss of elasticity and firmness of the skin due to the disorganisation and rarefaction of the collagen was studied in order to demonstrate the efficacy of the compositions of the invention.

Thus, the activity of the composition on the secretion of collagen I in culture medium by fibroblasts was evaluated.

The experimental module used consists of a culture of normal human fibroblasts up to the point of confluence. The cells are incubated with the composition at 0.05% (a concentration found to be non-cytotoxic to cells in the MTT test). Using the ELISA (Enzyme-Linked Immuno Sorbent Assay) method, which has the advantage of being easy to carry out, sensitive and specific, the type I collagen is metered into the culture medium.

### 3) Material and Method

First, fibroblast cultures were established by the explant method using samples of healthy Caucasian skin (abdominoplasty). The fibroblasts are cultured to the point of confluence, at 37° C. in a MEM medium (Minimum Essential Medium) containing 2 mM of L-glutamine and 10% foetal calf serum in a saturated humidity atmosphere with 5% CO2. Then, after separation using a buffered isotonic solution pH 7.2 of 0.1% trypsin and 0.02% EDTA, the cells are seeded into 96-well microplates (Falcon) at 10⁴ cells per well in the aforementioned medium. After 24 hours, this medium is replaced, for an incubation of 48 hours with the same medium without serum containing 0.15 mM sodium ascorbate (incubation medium) with or without the active ingredient complex to be tested.
One type of fibroblast was chosen for the study. These were 4th passage fibroblasts or fibroblasts from subculturing, representing cells having normal characteristics (fusiform or star-shaped appearance, good metabolic activity, good spreadability, etc.).
The quantity of type I collagen secreted in the medium after incubation with or without the active ingredient complex was then determined using an ELISA test. This method makes it possible to detect certain non-radioactive molecules at very low doses. A direct method was selected consisting of the absorption of the antigen (type I collagen) onto a solid phase (plastic of a microtitration plate reserved for ELISA). To this end, the incubation medium is collected and transferred to the wells of a microtitration plate. Incubation for 24 hours at +4° C. allows the adhesion of the collagen to the plastic. The plate is then rinsed with PBS (Phosphate Buffer Saline). After incubation for 24 h at +4° C. with serum albumin (2% in PBS) in order to avoid non-specific s-plastic antibody bonds, mouse anti-collagen I antibodies (Sigma) (dilution 1/2000) conjugated with an alkaline phosphatase (2 hours at TA) which in the presence of paranitrophenyl phosphate, will produce paranitrophenol which absorbs at 405 nm (1 hour at TA). The optical densities obtained are converted to ng of collagen by virtue of a calibration curve established under the same experimental conditions with type I collagen.

### 4) Results:

The results demonstrated that the composition used at 0.05% in the culture medium allows the fibroblasts to synthesis up to 6 times more type I collagen compared to the control (culture medium alone).
The cosmetic composition initiates and potentiates the synthesis programs of one of the major components of the extracellular matrix of the dermis, type I collagen. The composition therefore acts as an excellent “booster” for the reorganisation of the collagen fibres and thus makes it possible to improve the tonicity of the skin.

### EXAMPLE 9

### Evaluation of the Very Precious Cream

### 1) Aim:

Evaluation of the anti-wrinkle efficacy of the product.

### 2) Evaluation Criteria:

Analysis of the skin relief based on skin impressions.

### 3) Mode of Application:

The product is applied to the cleaned full face of 44 volunteers every evening for eight weeks. The detail of one mode of application is detailed below.

| | |
|---|---|
| D0-one week | Verification of inclusion and non- |
| | inclusion criteria. |
| | Signing of consent. |
| | Issuing of reference product. |
| D0-one week- | Application of the reference product to |
| D0 | the face twice daily (morning and evening) |
| | at home. |
| D0 | Creation of a skin impression. |
| | Issuing of the very precious cream. |
| D0-D28 | Application of the product to the face |
| | once daily (evening) at home, and |
| | application of the reference product to the |
| | face once daily (morning). |
| D28 | Verification of compliance with protocol. |
| | Evaluation of tolerance. |
| | Reporting of undesirable events and |
| | concomitant treatments. |
| | Creation of a skin impression. |
| D28-D56 | Application of the product to the face |
| | once daily (evening) at home, and |
| | application of the reference product to the |
| | face once daily (morning). |
| D56 | Verification of compliance with protocol. |
| | Return of product and of tracking sheet. |
| | Evaluation of tolerance. |
| | Reporting of undesirable events and |
| | concomitant treatments. |
| | Response to cosmetic acceptability |
| | questionnaire. |
| | Creation of a skin impression. |

### 4) Results:

After just four weeks, a 16% reduction in the number of wrinkles and a 26% reduction in the total wrinkled surface area (average improvement measured across 25 volunteers).

After eight weeks of use, the efficacy is felt. The results obtained are described below.

| | |
|---|---|
| The skin is firmer | 89% |
| The skin is smoother | 87% |
| The skin appears regenerated | 89% |
| Wrinkles are improved | 85% |

1. A cosmetic or dermatological composition, characterised in that it comprises:
(i) at least plant oils which are rich in omega-3 and/or in omega-6, selected from the group comprising echium, camelina, evening primrose and borage oils, which plant oils are present in a proportion which makes it possible to obtain a ratio of omega-3 to omega-6 in said composition of between 1 and 5,
(ii) at least one mineral, preferably in salt form, selected from the group comprising sodium, calcium and magnesium, preferably magnesium,
(iii) at least one trace element selected from the group comprising zinc, copper and iron, preferably from the group comprising zinc and copper,
(iv) at least vitamin C or a derivative thereof, and
(v) at least one essential oil, which has anti-free radical properties.

2. The composition according to claim 1, in which said essential oil is a Helichrysum essential oil (Helichrysum italicum).

3. The composition according to claim 2, in which said composition comprises a content of Helichrysum essential oil of between 0.05% and 5% of the total weight of the composition, preferably between 0.05 and 2.5%.

4. The composition according to claim 1, in which said composition comprises echium, camelina, evening primrose and borage oils.

5. The composition according to claim 1, in which said composition comprises a content of:
a) echium oil of between 0.1% and 10% of the total weight of the composition, preferably between 0.1 and 5%, and/or
b) camelina oil of between 0.1% and 10% of the total weight of the composition, preferably between 0.1 and 5%, and/or
c) borage oil of between 0.1% and 10% of the total weight of the composition, preferably between 0.1 and 5%, and/or
d) evening primrose oil of between 0.1% and 10% of the total weight of the composition, preferably between 0.1 and 5%.

6. The composition according to claim 1, in which said composition comprises a content of:
a) magnesium of between 0.001% and 5% of the total weight of the composition, preferably between 0.001 and 1%, and/or
b) copper of between 0.001% and 5% of the total weight of the composition, preferably between 0.001 and 1%, and/or
c) zinc of between 0.001% and 5% of the total weight of the composition, preferably between 0.001 and 1%.

7. The composition according to claim 1, in which said composition comprises zinc, copper and magnesium.

8. The composition according to claim 1, in which said composition comprises a content of vitamin C or one of its derivatives of between 0.5% and 10% of the total weight of the composition, preferably between 0.5% and 5%.

9. The composition according to claim 1, in which said composition comprises a vitamin C derivative, preferably Ascorbic Acid 2-Glucoside (or O-α-D-glucopyranosyl-(1→2)-L-ascorbic acid).

10. The composition according to claim 1, in which said composition furthermore comprises at least one other active ingredient selected from the group comprising refining, restructuring, anti-inflammatory, hydrating and antiseptic agents for the skin.

11. The composition according to claim 10, in which said composition comprises a content of said active ingredient of between 1 and 20% of the total weight of the composition, preferably between 1 and 10%.

12. A cosmetic care method comprising a step of applying to the skin of a subject an effective quantity of a composition according to claim 1.

13. A cosmetic use of a composition according to claim 1 for carrying out a deep treatment which stimulates the microcirculation and the production of energy in order to revitalise the cells and to accelerate their renewal, to protect the collagen and to combat pigmentation marks.
